(19) United States
(12) Patent Application Publication
(10) Pub. No.: US 2012/0319311 A1
(43) Pub. Date: Dec. 20, 2012

(54) UV STERILIZATION CHAMBER FOR A HUMIDIFIER

(75) Inventors: Francis C Nutter, Charlton, MA (US); Zhijing Wang, Boxborough, MA (US); Robert Zhao, Shenzhen (CN); Edisen Lu, Shenzhen (CN); Yongming Sun, Shenzhen (CN); Sean Wang, Shenzhen (CN); Christopher S. Kanel, Hudson, NY (US)

(73) Assignee: KAZ USA, INC, Southborough, MA (US)

(21) Appl. No.: 13/002,598

(22) PCT Filed: Oct. 20, 2010
(86) PCT No.: PCT/US10/53344

§ 371 Date: Sep. 10, 2012

(30) Foreign Application Priority Data
Oct. 20, 2009 (US) 61279445

(51) Int. Cl. F24F 6/02; A61L 2/10

(52) U.S. Cl. 261/72.1; 422/24; 250/437

(57) ABSTRACT

A UV sterilization chamber is provided to a humidifier having a water reservoir, a humidifying element and a pathway for directing water provided by the water reservoir to the humidifying element of the humidifier. The pathway is provided in a humidifier base, over which a humidifier enclosure is removably placed. The UV sterilization chamber includes a serpentine portion of the pathway and a UV radiation source positioned for illuminating the portion of the pathway with UV light. Upwardly-directed projections in the base border a perimeter of the pathway; and a downwardly-directed projection of the UV radiation source extends between the upwardly-directed projections when the enclosure is mated with the base, thereby locating the UV radiation source over the portion of the pathway. A switch disables the UV radiation source when the enclosure is removed from the base.

### FIELD OF THE INVENTION

The present invention relates generally to humidifying devices, and more specifically, to an ultraviolet (UV) sterilization chamber suited for use in an evaporative humidifier.

### BACKGROUND OF THE INVENTION

Humidifying devices, or humidifiers, are used to moisturize the ambient air in a room, most commonly, a single room of a home. Evaporative (or wick) humidifiers, for example, utilize a reservoir of water, from which the wick draws moisture, and a fan forces air through and/or past the wick, thereby picking up moisture in the air as it is directed through a spout or vent into the room.

It is well known that ultraviolet (UV) irradiation may be used to reduce or eliminate the various minerals, microorganisms, and other contaminants in water. In particular, UV irradiation has been provided in an evaporative humidifier to sterilize the water supply to the wick. See, for example, the evaporative humidifier as described in the commonly-owned U.S. Pat. No. 7,513,486, (“the '486 Patent”), the entire contents of which are incorporated by reference herein.

In order for sterilization to be achieved, the water being provided to the wick must be exposed to UV irradiation for a sufficiently long period of time. In the '486 Patent, a UV lamp is provided in a disinfection unit and the lamp is surrounded with a helical ramp around which the supplied water travels prior to reaching the wick. By traveling along the extended path provided by the helical ramp as it is irradiated by the UV lamp, the supplied water receives sufficient irradiation for effective sterilization. However, this unit is relatively complex to fabricate, and subjects the quartz tube to the risk of being directly contacted by the water as a result a failure of the unit.

### SUMMARY OF THE INVENTION

According to an embodiment of the present invention, a novel UV sterilization chamber is disclosed for use in a humidifier having a fellable water reservoir, a humidifying element and a pathway for directing water provided by the fillable water reservoir to the humidifying element of the humidifier. The pathway is provided in a base of the humidifier, over which an enclosure of the humidifier is removably placed. The sterilization chamber includes a portion of the pathway and a UV radiation source that is provided in the enclosure and positioned over the portion of the path way for illuminating the portion of the pathway with UV light. Upwardly-directed projections are provided in the base that border a perimeter of the pathway; and a downwardly-directed projection of a housing for the UV radiation source is configured to extend between the upwardly-directed projections when the enclosure is mated with the base in order to locate the UV radiation over the portion of the pathway to be illuminated. The downwardly-directed projection of the housing includes a radiation window for emitting the UV radiation. The housing also includes a switch configured to disable the UV radiation source when the enclosure is removed from the base

The housing further preferably includes a reflector for reflecting UV light emitted from a UV lamp downwardly through the radiation window. In addition, the illuminated portion of the pathway preferably comprises a serpentine pathway, which increases the time of travel of the water through the pathway and thereby the time of exposure to the UV radiation as a sterilant.

By configuring the humidifier to include a base with a removable enclosure in which the water pathways are primarily confined to the base unit, and in which the UV radiation source is easily and readily separated and removed from these water pathways, fabrication and cleaning of the humidifier are simplified over prior art systems.

### BRIEF DESCRIPTION OF THE DRAWINGS

The foregoing and other features of the present invention will be more readily apparent from the following detailed description and drawings of illustrative embodiments of the invention in which:

FIG. 1 is a perspective cutaway view of a humidifier including a UV sterilization chamber in accordance with a preferred embodiment of the present invention;

FIGS. 2A-2D are exploded perspective views of component sections of the humidifier of FIG. 1;

FIG. 3 is a top view of a base of the humidifier of FIG. 1;

FIG. 4 is bottom view of the humidifier of FIG. 1, with the base of FIG. 3 having been removed;

FIG. 5 is a front exploded view of the humidifier of FIG. 1, with cutaway portions showing elements of the UV sterilization chamber in a separated state; and

FIG. 6 is a front exploded view of the humidifier of FIG. 1, with cutaway portions showing elements of the UV sterilization chamber in an engaged state.

Like reference numerals are used in the drawing figures to connote like components of the humidifier.

### DETAILED DESCRIPTION OF THE PREFERRED EMBODIMENTS

A preferred embodiment of an ultraviolet (UV) sterilization chamber for a humidifier according to the present invention is described below. This embodiment is provided for the purpose only of illustrating principles of the present invention, and should not be interpreted as limiting the invention in any way beyond the scope of the claims and their equivalents.

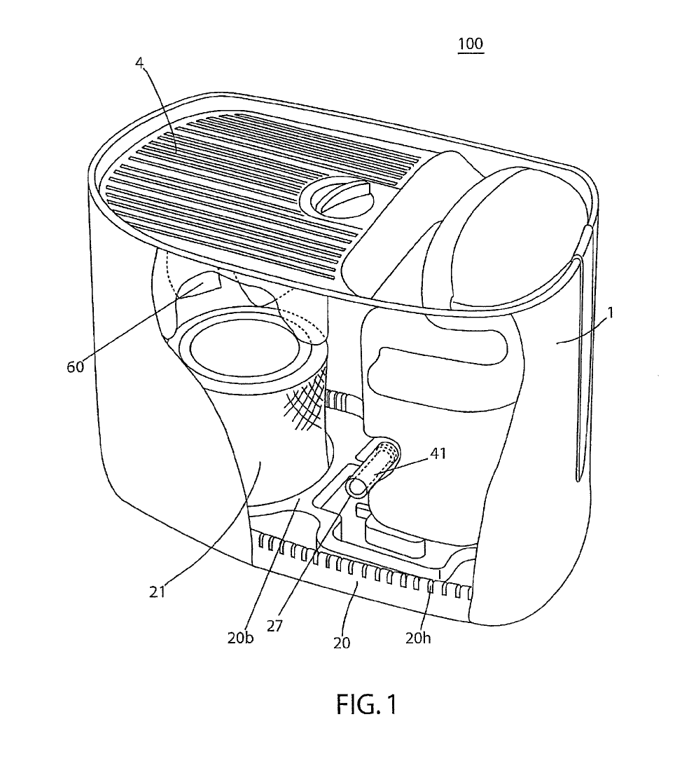

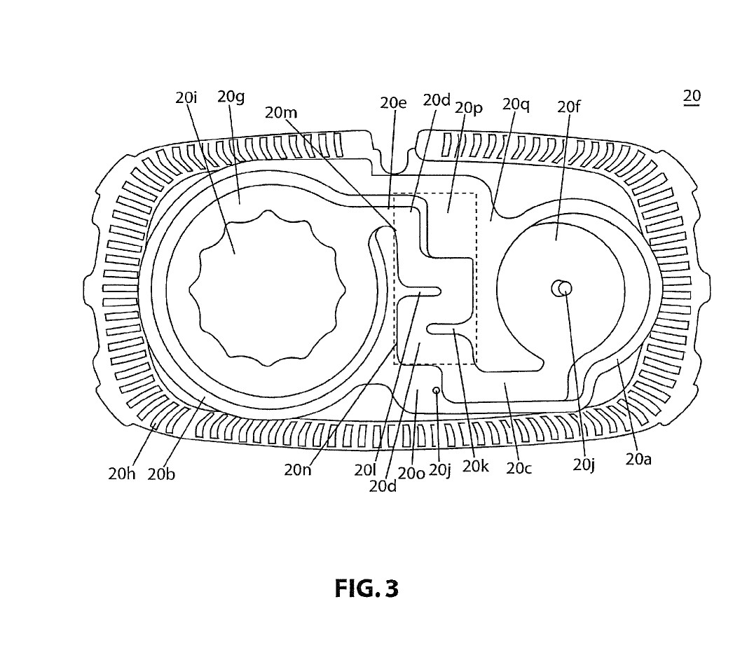

Referring to FIG. 1, a humidifier 100 includes a body 1 carried on a base 20. The base 20 has a molded ring 20a (see, e.g., FIG. 3) for carrying a water reservoir 50, as well as a molded ring 20b for carrying a wicking filter 21. The filter 21 is preferably formed from several layers of “expanded” cellulose (paper), each layer of cellulose being slit and stretched into a non-raveling open mesh and then mechanically or adhesively adhered to the other layers. This filter design enables water to be fully absorbed while maintaining air flow through the cellulose layers in order to achieve a maximum evaporation rate. Expanded cellulose filters of this type may be obtained, for example, from Columbus Industries, Inc. of Columbus, Ohio.

Water exiting the water reservoir 50 is directed from a compartment 20f defined by molded ring 20a along a series of molded paths 20c, 20d and 20e to a compartment 20g defined by the molded ring 20b and a molded insert 20i (see, e.g., FIG. 3). The filter 21 rests within the compartment 21, and absorbs water by capillary action (“wicking”) to moisten a substantial volume of the filter. The moisture is evaporated from the filter with the assistance of a fan and control unit 60, which draws air through vents 20h of the base 20 and around the filter 21 to be exhausted through a grill 4 positioned in an opening 1 a in the upper surface of the body 1.

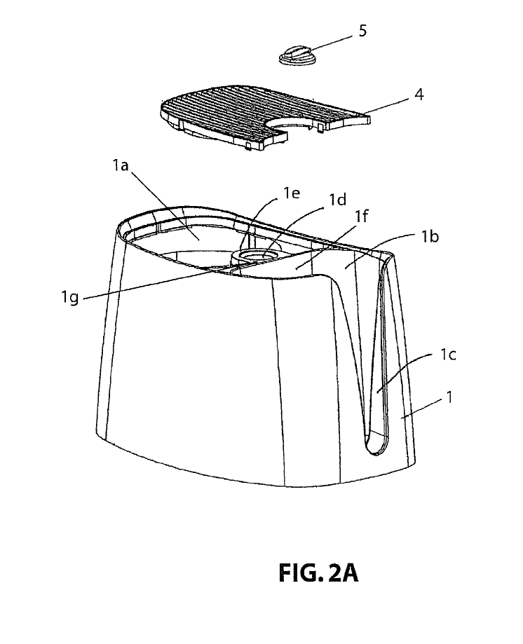

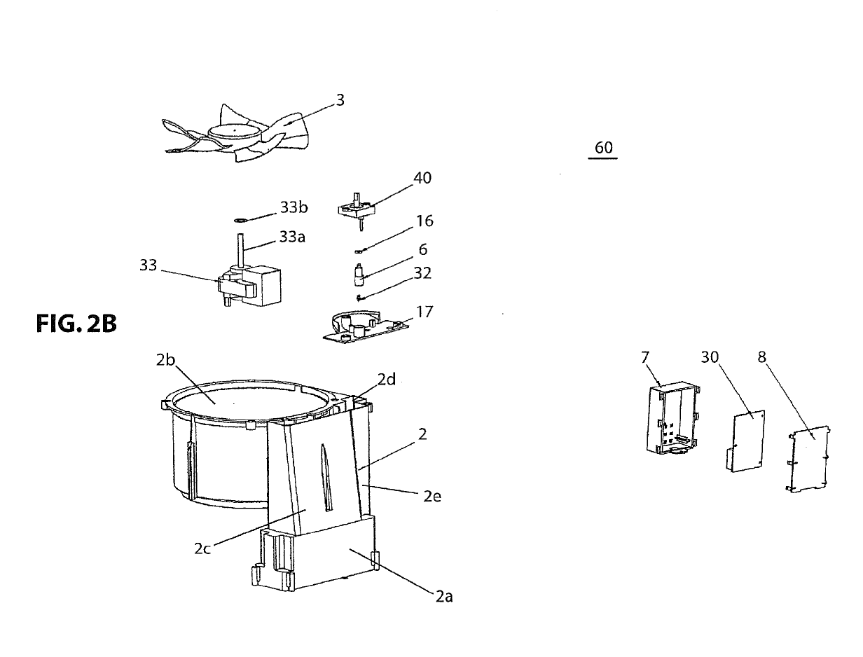

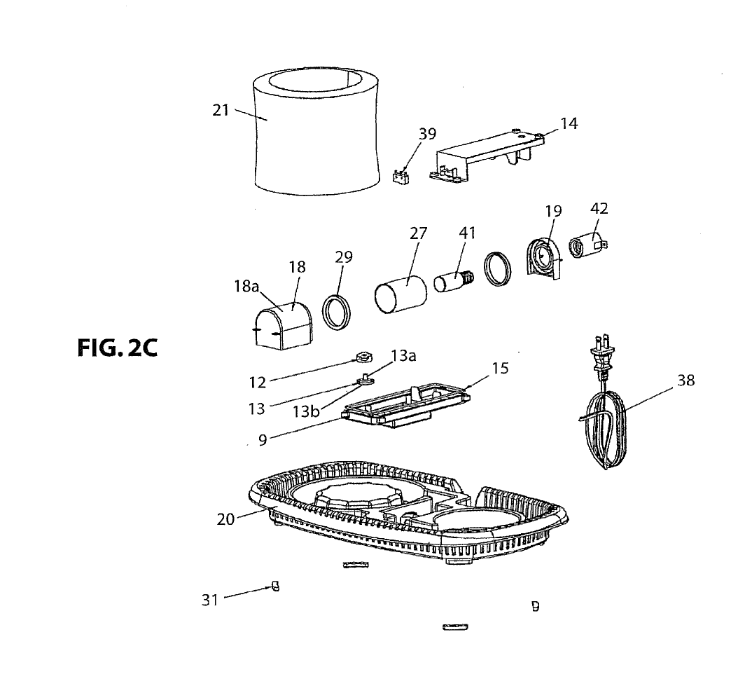

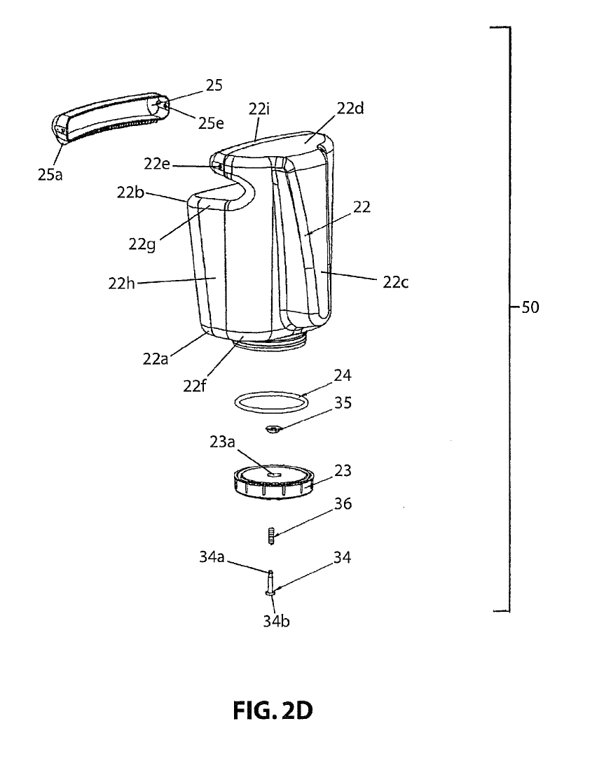

With reference to FIGS. 2B and 2C, a UV lamp 41 is positioned within a quartz glass tube 27 in a lower cavity of a motor cavity 2 of the fan and control unit 60. The UV lamp 41 may preferably be a filament-type, 4-watt UV lamp that produces UV radiation having wavelengths in the UV-C (or shortwave) range of 200 to 280 nanometers, which may be obtained for example from Osram Sylvania of Danvers, Mass. Radiation in the UV-C range has been proven effective as a mutagen in the destruction of the DNA of micro-organisms such as pathogens, viruses, bacteria and molds, thereby preventing their reproduction. See, e.g., Philip W. Brickner, MD et al., “The Application of Ultraviolet Germicidal Irradiation to Control Transmission of Airborne Disease: Bioterroism Countermeasure,” Public Health Reports, Vol. 118, March- April 2003, pp. 99-119, which is incorporated by reference herein.

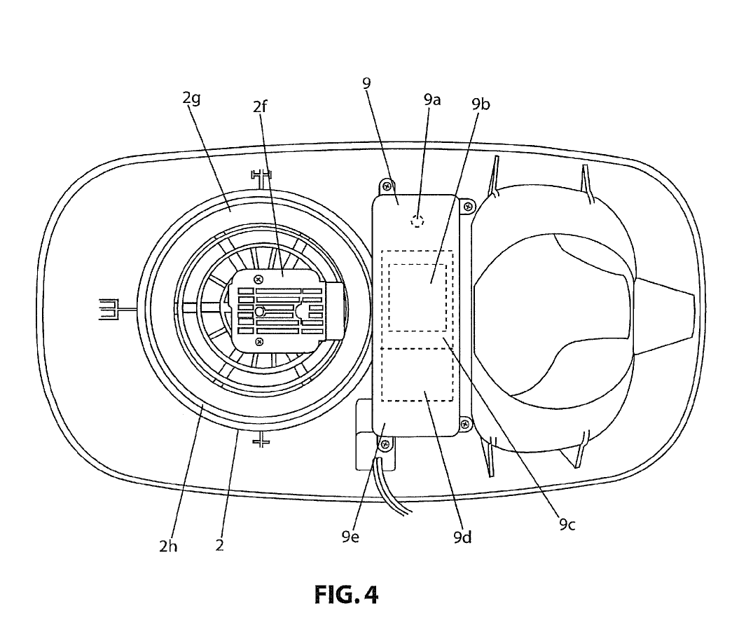

A UV bracket 9 is fastened at a bottom surface of the cavity to retain the UV lamp 41 and quartz glass tube 27 within the cavity, and includes an aperture 9b (see, e.g., FIG. 4) permitting UV light from the UV lamp 41 to be downwardly directed toward the molded path 20d to irradiate water in the molded path 20d, through which water provided by the water reservoir 50 is directed to the filter 21.

With reference to FIG. 2A, an exploded diagram further illustrates the body 1, the grill 4 and a knob 5. The body 1 may be formed in any number of shapes, and as shown is preferably formed as a substantially rectangular box made of molded plastic. The knob 5 attaches to a switch 40 of the fan and control unit 60 (see FIG. 2B) at an aperture 1d in a ring 1e located at the upper surface of the body 1. In addition, an aperture 1g is provided in the ring 1e adjacent to the aperture 1d. The ring 1e is integrally formed with a vertical rib 1f of the body 1.

The body 1 further includes a cavity 1b for receiving the water reservoir 50, and a slot 1c through which an indicator portion 22c of the tank member 22 of the reservoir 50 protrudes in order to provide a visual indication of the water level in the reservoir 50.

With reference to FIG. 2B, the fan and control unit 60 includes a fan blade 3 which is installed on a drive shaft 33a of motor 33 and retained by a circlip 33b or other suitable fastener. The fan blade 3 may preferably be a five-bladed, seven-inch axial blade design, or any other suitable design.

The fan motor 33a is mounted in a motor bracket 2f that is mounted to base member 2g within a bowl 2b of the motor bracket 2 (see, e.g., FIG. 4), and is controlled by conventional control circuitry that is mounted on a printed circuit board (PCB) 30 via a rotary switch 40. The fan motor 33a may preferably be a 1380 RPM, single-phase shaded-pole 120 VAC motor (conventionally referred to as a “universal” motor), or a motor of other suitable design. Alternatively, the fan motor 33a may be a 1380 RPM, single-phase shaded-pole motor operating at 220-240 VAC. Motors of these types may be obtained, for example, from Wolong Electric Group, Ltd. Of Shangyu (Zhejiang province), China.

The rotary switch 40 is preferably a multiple position switch that, in combination with the control circuitry, enables a user to operate the fan at several selectable fan speeds.

The base member 2g may preferably include a rim 2h which is configured to locate the upper surface of the cylindrical filter 21 against the base member 2h. Similarly, molded ring 2b locates a bottom surface of the cylindrical filter 21 against a bottom surface of the compartment 20g. In this configuration, the fan operates to draw air through vents 20h in the base 20 toward an external surface of the cylindrical filter 21 through to an interior of the filter 21 to be exhausted through the grill 4 positioned in the opening 1a in the upper surface of the body 1.

With reference to FIG. 2B, the PCB 30 is housed in a PCB box 7 having a PCB box cover 8, which is housed within a control compartment 2c of the motor bracket 2. The PCB 30 also controls the operation of the UV lamp 41, as will be further described herein, and a light emitting diode (LED) 32, which is housed in a lamp shade 6. The rotary switch 40 and the LED 32 in its lamp shade 6 are mounted to a rotary switch cover 17, which is fastened to one or more of the body 1 and motor bracket 2 so that the rotary switch 40 protrudes through the aperture 1d and the lamp shade 6 extends through the aperture 1g. The lamp shade 6 is firmly fitted within the aperture 1g by means of an O-ring 16. The LED 32, in combination with the control circuitry, is preferably configured to indicate one or more of “power on” condition and/or a “maintenance needed” condition (for example, filter or UV lamp replacement) for the humidifier 100. The LED 32 may preferably be configured to emit several colors of light under the control of the PCB 30 in order to indicate different operating conditions of the humidifier 100.

A conduit 2e of the motor bracket 2 reaches to the base 20 and provides a path for a conventional power cord 38 (see, e.g., FIG. 2C) to extend externally from the humidifier 100.

With reference to FIG. 2C, the base 20 is shown with reference to the filter 21 and a UV lamp unit including the UV lamp 41 and the quartz glass tube 27. As shown, the base 20 is preferably provided with rubber feet 31 at each corner of the base 20 for stable placement of the base 20 on an operating surface.

The lamp 41 and the quartz glass tube 27 are housed in a UV box 18. A UV lamp holder electrically interconnected to the PCB 30 (see FIG. 2B), is inserted through an aperture of a UV box cover and receives the UV lamp 41. In order to guard against the ingress of moisture to the vicinity of UV lamp 41, seal pads 29 are provided at either end of the quartz glass tube 27 to seal the quartz glass tube 27 against end faces of the UV box 18 and UV cover 19. The assembled UV box 18 and UV box cover 19 are enclosed by a cap 14 and the UV bracket 9, and the entire assembly is inserted into a UV lamp unit compartment 2a. The UV bracket 9 is sealed to the UV lamp unit compartment 2a by means of an O-ring 15. As illustrated, the UV box 18 includes an upper member 18a that is arced (preferably along a parabolic profile). An inner surface of the upper member 18a is preferably provided with a reflective material, such that the inner surface of the upper member 18a is effective to direct UV light radiated by the UV lamp 41 downwardly to irradiate water in the molded path 20d of FIG. 3. When a filament-type, 4-watt UV-C lamp is provided in the UV box 18, the upper member 18a is provided with the reflective material and the UV lamp 41 is positioned less than one inch above a surface of the water in the molded path 20d, it is possible to achieve a germ-killing effectiveness of 99.99%.

The cap 14 and UV bracket 9 further mount a microswitch 39, fixed chip 12 and push rod 13 to the UV lamp unit. The microswitch 39 is also electrically interconnected to the PCB 30. The push rod 13 is preferably fabricated from silicone rubber, and a rod portion 13a extends through an aperture in the fixed chip 12. The rod portion 13a of the push rod 13 is positioned for actuating the microswitch 39, and a base portion 13b of the push rod 13 is placed in contact with the UV bracket 9 over an aperture 9a (see, e.g., FIG. 4) positioned to be flexibly displaced by a pin 20j of the base 20 (see, e.g., FIG. 3). When the body 1 of the humidifier 100 is placed onto the base 20, the pin 20j extends through the aperture 9a to flexibly displace the base portion 13b of the push rod 13, such that the rod portion 13a engages the microswitch 39 to switch the microswitch 39 to an electrically closed position. In this position, the UV lamp is powered when the rotary switch 41 is switched to an operating position. When the body 1 is removed from the base 20, the pin 20j exits the aperture 9a and the rod portion 13a of the push rod 13 retracts to switch the microswitch 39 to an electrically open position. In this position, one or more of the UV lamp and or the fan unit are inoperative regardless of the position of the rotary switch 41.

With reference to FIG. 2D, the water reservoir 50 includes a tank member 22 which may be formed in any of a variety of volumetric shapes that may be suitable for coupling the water tank 100 to the humidifier. A principal portion of the tank member 22 has an approximately trapezoidal lower face 22a with rounded vertices, and extends upwardly along a longitudinal direction of the portion with a continuously expanding cross-section that reaches its maximum cross-sectional area at an upper face 22b. Respective perimeter edges 22f, 22g of the lower face 22a and upper face 22b are also rounded, and join side walls 22h, a rear wall 22j and a front wall (not shown) of the principal portion to the lower face 22a and upper face 22b, respectively.

The tank member 22 further includes a handle portion 22d that extends upwardly from a rear portion of the upper face 22b and forward from this portion, but not completely across the upper face 22b. Over the portion of the upper face 22b that the handle portion 22d extends, it has essentially the same cross-section as this portion of the upper face 22b.

A front region 22i of the handle portion 22d is slightly inset to receive a tank cover 25. The tank cover 25 preferably includes a gripping portion 25a for gripping by a human hand, and recesses 25e which mate with projections 22e on the tank member 22 in order to positively locate the tank cover 25 over the front region 22i of the handle portion 22d. The tank cover 25 in addition may be optionally bonded to the handle portion 22d with a conventional adhesive.

An indicator portion 22c of the tank member 22 extends rearwardly from the rear face 22k of the tank member 22 and handle portion 22d As illustrated in FIG. 1, a depth of the indicator portion 22c increases in a downward vertical direction, while a width of the indicator portion 22c decreases in the downward vertical direction. The indicator portion 22c of the water tank 100 is configured for example to extend through a wall of the humidifier to provide a visual indication to the user regarding the fluid level in the tank member 22,

At the lower face 22a, a round, externally threaded opening 22j is provided for receiving a cap 23, which has an inner thread for mating with an outer thread of the threaded opening 22j. The cap 23 is provided with an O-ring 23 for sealably securing the cap 23 to the threaded opening 22j of the tank member 22.

A valve bar 34 is positioned within a central aperture 23a of the cap 23. A downward force is exerted on the valve bar 34 by a spring 36, which is fitted over the valve bar 34 and between a flange 34b of the valve bar 34 and the upper surface of a spring cup (not shown) of the cap 23. A rubber valve member 35 is lockably fitted to a groove 34a of the valve bar 34, and serves to restrict further downward movement of the valve bar 24 when the valve member 35 is interferingly pulled by the force of the spring 36 against the central aperture 23a. When inserted into the compartment 20g of the base 20, a post 20j (see, e.g., FIG. 3) positioned at the center of the compartment 20g is pressed against a bottom surface of the flange 34b of the valve bar 34, thereby raising the valve member 35 above the central aperture 23a and permitting fluid in the water tank 100 to flow around the valve 34a and through the central aperture 23a for delivery to the wick of the humidifier.

With reference to FIG. 3, the base 20 as previously described includes the compartment 20f defined by the molded ring 20a, the compartment 20g defined by the molded ring 20b and the molded insert 20i, and the molded paths 20c, 20d and 20e which interconnect and enable water to flow from the compartment 20f to the compartment 20g. Molded path 20d is positioned near the center of the base 20, and has a serpentine shape defined by vanes 20k and 20l. The serpentine path carries water supplied by the path 20c near a front side of the base 20 to the path 20d near the rear side of the base 20. Each of paths 20c and 20e carry water across the base 20 to the serpentine path 20d.

In FIG. 3, the serpentine path 2d is shown with a region 20n, With reference to FIG, 4, the region 20n corresponds to a position occupied by a first rectangular projection 9c of the bracket 9 when the body 1 of the humidifier 100 is placed onto the base 20. As the first rectangular projection 9c includes the aperture 9b through which UV light from the UV lamp 41 is emitted, water carried within the serpentine path 2d is thereby irradiated. By following the serpentine path defined by the molded path 2d, the amount of time required for water to be carried along the path is increased, thereby increasing the time that the water is exposed to the UV light and increasing the effectiveness of the UV sterilization.

With reference to FIG. 4, the bracket 9 includes the first rectangular projection 9c and a second rectangular projection 9d that extend downwardly from a base bottom surface 9e of the bracket 9. When the body 1 of the humidifier 100 is placed on the body 20, the first rectangular projection is positioned over the region 20n of FIG, 3, and the second rectangular projection is positioned over a region 20m of FIG. 3. As further shown in FIG. 3, the perimeter of regions 20m, 20n is bordered by molded projections 20o and 20q. The region 20m further includes a pad 20p, which also defines a portion of the serpentine path 20d.

Upper surfaces of each of the vanes 20k, 20l and the pad 20p lie below the upper surfaces of the molded projections 20o, 20q. As a result, when the body 1 of the humidifier 100 is placed on the body 20, the first and second projections 9c, 9d extend below the upper surfaces of the molded projections 20o, 20q into the regions 20n, 20m, respectively. In this manner, the molded projections 20o, 20q operate to properly locate the first and second projections 9c, 9d (and the UV lamp 41 positioned above the aperture 9b) with respect to the serpentine path 20d, and thereby assist in confining the emitted UV light to the serpentine path.

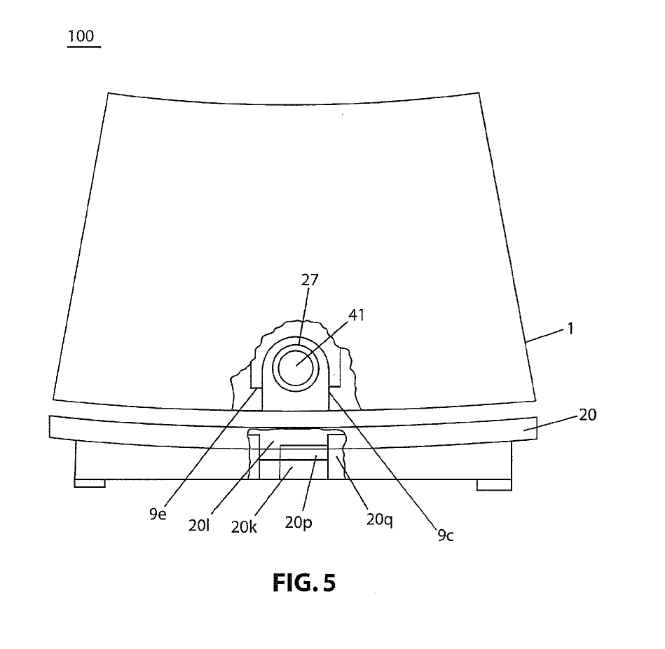

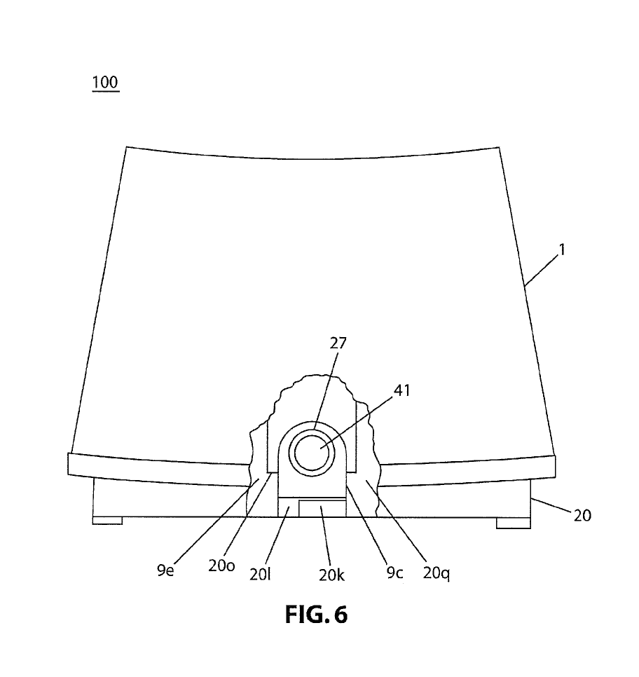

These features can be further illustrated as follows. FIGS. 5 and 6 each show the body 1 and the base 20 of the humidifier 100 in partially broken views that make visible elements of the disclosed UV sterilization chamber. FIG. 5 illustrates the humidifier 100 in a state in which the body 1 has been separated from the base, and FIG. 6 illustrates the humidifier 100 in a state in which the body 1 has been placed onto the base 20. With reference to FIG. 6, it can be seen that, when the body 1 is placed onto the base 20, the first projection 9c is directed between the molded projections 20o, 20q until portions of the base bottom surface 9e rest on the upper surfaces of the molded projections 20o, 20q. In this position, a bottom surface of the first projection 9c extends below the top surfaces of the molded projections 20o, 20q and the top surface of the pad 20p, in proximity to the top surfaces of the vanes 20k and 20l. Side surfaces of the first projection 9c substantially abut side surfaces of the molded projections 20o, 20q, thereby locating the first projection 9c over the serpentine path 2d in a substantially fixed position.

While the invention has been particularly shown and described with reference to preferred embodiments thereof, it will be understood by those skilled in the art that various changes in form and details may be made therein without departing from the spirit and scope of the invention. For example, the first and second rectangular projections of the UV bracket may be made in any of a variety of shapes determined as a function of the humidifier design (in particular, as a function of the corresponding molded projections of the base. In addition, the serpentine path may include any of a variety of configurations of folded paths, and the serpentine path and lamp unit may be positioned at any of a variety of positions relative to a footprint of the base.

Accordingly, the invention is to be limited only by the scope of the claims and their equivalents.

What is claimed is:

1. A humidifier comprising:
a finable water reservoir;
a base including a pathway for directing water provided by the fillable water reservoir to a humidifying element of the humidifier; and
an enclosure configured to be fixedly and removably mated with the base, the enclosure including a UV radiation source,
wherein, when the enclosure is mated with the base, the UV radiation source is positioned over at least a portion of the pathway for illuminating the portion of the pathway with UV light.

2. The humidifier of claim 1, wherein the portion of the pathway is configured as a serpentine pathway.

3. The humidifier of claim 1, wherein the UV radiation source comprises: a UV lamp; and a housing for housing the UV lamp.

4. The humidifier of claim 3, wherein the housing further comprises:
a reflector for directing UV light generated by the lamp downwardly toward the portion of the pathway.

5. The humidifier of claim 3, wherein the UV radiation source further comprises:
a transparent tube sealably mounted in the housing and surrounding the UV lamp.

6. The humidifier of claim 5, wherein the transparent tube is a quartz glass tube.

7. The humidifier of claim 3, further comprising:
upwardly-directed projections in the base bordering a perimeter of the pathway; and
a downwardly-directed projection of the housing, the downwardly directed projection being configured to extend between the upwardly-directed projections when the enclosure is mated with the base to locate the UV lamp over the portion of the pathway for illuminating the portion of the pathway with the UV light.

8. The humidifier of claim 7, wherein the downwardly-directed projection further comprises an aperture configured for emitting UV light generated by the UV lamp.

9. The humidifier of claim 3, further comprising
a control unit for controlling the operation of the UV lamp, wherein:
the housing further comprises a switch, and
the control unit is configured to power the UV lamp when the switch is closed and to depower the UV lamp when the switch is open.

10. The humidifier of claim 3, wherein:
the base further comprises a post, the post being configured to the close the switch when the enclosure is mated with the base.

11. The humidifier of claim 9, wherein the switch is configured to open when the enclosure is removed from the base.

12. The humidifier of claim 1, wherein the humidifying element comprises:
a compartment for receiving water from an exit of the pathway;
a wicking filter positioned for absorbing water from the compartment;
a fan unit provided in the enclosure and configured to draw air through the filter for evaporating the water absorbed by the wicking filter.

13. The humidifier of claim 12, wherein the base further comprises one or more vents for admitting air into the enclosure.

14. The humidifier of claim 12, wherein the enclosure further comprises an opening in an upper surface of the enclosure adjacent to the fan that is configured for exhausting the air drawn by the fan.

15. The humidifier of claim 1, wherein the base further comprises a compartment for refillably receiving water from the fillable water reservoir and providing the received water to an entrance of the pathway.

16. A sterilization chamber for a humidifier, the chamber comprising:
a serpentine pathway configured to be provided in a base of the humidifier, the pathway being configured to direct a flow of water toward a humidifying element of the humidifier; and
a UV radiation source configured to be provided in an enclosure of the humidifier, the enclosure being configured to be fixedly and removably mated with the base;
wherein, when the enclosure is mated with the base, the UV radiation source is positioned in proximity to a portion of the serpentine pathway for illuminating the portion of the serpentine pathway with UV light.

17. The sterilization chamber of claim 15, wherein the UV radiation source comprises:
a UV lamp; and
a housing for housing the UV lamp.

18. The humidifier of claim 17, wherein the housing further comprises:
a reflector for directing UV light generated by the lamp downwardly toward the portion of the pathway.

19. The sterilization chamber of claim 17, further comprising:
upwardly-directed projections to be provided in the base bordering, the upwardly-directed projections bordering a perimeter of the pathway; and
a downwardly-directed projection of the housing, the downwardly-directed projection being configured to extend between the upwardly-directed projections when the enclosure is mated with the base to locate the UV lamp over the portion of the pathway for illuminating the portion of the pathway with the UV light.

20. The sterilization chamber of claim 19, wherein the downwardly-directed projection further comprises an aperture configured for emitting UV light generated by the UV lamp.

21. The sterilization chamber of claim 17, wherein:
the housing further comprises a switch; and
the pathway farther comprises a post configured for operating the switch,
whereby when the UV radiation source is removed from proximity to the portion of the serpentine pathway, the switch is switched from a closed state to an open state.

22. A method for sterilizing water for delivery to a humidifying element of a humidifier, the method comprising the steps of
providing a serpentine pathway between a water-supplying compartment and a water-receiving compartment of the humidifier;
providing upwardly-directed projections that border a perimeter of the pathway;
providing a UV radiation source in a housing having a downwardly-directed projection; providing a radiation window in the downwardly-directed projection;
positioning the downwardly-directed projection between the upwardly-directed projections to locate the radiation window over the serpentine pathway;
directing a water flow through the serpentine pathway; and
directing UV radiation from the UV radiation source through the radiation window, thereby illuminating the serpentine pathway with UV radiation and sterilizing the water flow.

23. A method for sterilizing water for delivery to a humidifying element of a humidifier, the method comprising the steps of:
providing a serpentine pathway between a water-supplying compartment and a water-receiving compartment of the humidifier;
providing a UV radiation source in a housing having a downwardly-directed radiation window;
positioning the radiation window over the serpentine pathway; directing a water flow through the serpentine pathway; and
directing UV radiation from the UV radiation source downwardly through the radiation window, thereby illuminating the serpentine pathway with UV radiation and sterilizing the water flow.

24. The method of claim 23, further comprising the steps of:
providing the serpentine pathway in a base of the humidifier;
providing the UV radiation source and housing in an enclosure configured to be removably mated with the base;
wherein the radiation window is positioned over the serpentine pathway when the enclosure is mated with the base.
